(19) United States
(12) Patent Application Publication
(10) Pub. No.: US 2013/0018278 A1
(43) Pub. Date: Jan. 17, 2013

(54) Blood Draw Catheter Top

(71) Applicant: Elsa Leon, Phoenix, AZ (US)

(72) Inventor: Elsa Leon, Phoenix, AZ (US)

(21) Appl. No.: 13/467,701

(22) Filed: May 9, 2012

Related U.S. Application Data
(60) Provisional application No. 61/572,359, filed on Jul. 13, 2011.

(51) Int. Cl. A61B 5/153

(52) U.S. Cl. 600/573

(57) ABSTRACT

The invention relates to a blood draw catheter top having a cap and tube portion. The top is a phlebotomy tool specifically designed to introduce an intravenous catheter into blood collection. The top is intended to provide easier and less painful blood draw experiences by facilitating multiple draws from a single stick site on a patient. Placed over a needle, the top, via internal threads, controls the flow of blood to allow blood sample collection.

### CLAIM OF PRIORITY

This patent application claims priority under 35 USC 119 (e) (1) from Provisional Patent Application Ser. No. 61/572,359 filed Jul. 13, 2011, of common inventorship herewith entitled, “Blood Draw Catheter Top.”

### FIELD OF THE INVENTION

The present invention pertains to the field of blood supplies, and more specifically to the field of phlebotomy tools used with blood collection.

### BACKGROUND OF THE INVENTION

The prior art has put forth several designs for blood collection devices. Among these are:

U.S. Pat. No. 7,521,022 to Franz Konrad describes a blood receiving device comprising a holding container for a holding vessel, a collecting tube, a needle, a needle holder, and an adjustable covering element.

U.S. Pat. No. 7,396,342 to Shih-Chun Wang, Kiwi Yuan and Jia-Ming Chang describes a safety syringe for taking blood comprising a hollow barrel, a needle hub, a cap and an enclosure. The safety syringe safely retracts a used needle back into a barrel and prevents health workers from being injured or infected by used needles.

US Patent 2008/0262421 to Steven Schraga describes a retractable fluid collection device for use in collecting blood. This invention comprises a holder having a double ended needle that automatically retracts into the holder when the health worker closes a cap over the open end of the holder.

None of these prior art references describe the present invention.

### SUMMARY OF THE INVENTION

It is an object of the present invention to provide a blood collection device which introduces an intravenous or IV catheter into a blood vessel for blood collection.

### BRIEF DESCRIPTION OF THE DRAWINGS

FIG. 1 is a functional perspective view showing a blood container and needle being inserting into the cap containing an IV needle.

FIG. 2 is a close up view of the cap that is placed over syringe or needle insertion hole in an IV.

FIG. 3 is a detailed diagram of the cap and tube portions of the invention.

### DETAILED DESCRIPTION OF THE INVENTION

Phlebotomy is the act of drawing or removing blood from the circulatory system through an incision or puncture to obtain a sample for analysis and diagnosis. This procedure is performed by a technician known as a phlebotomist who takes the blood from a vein on the back of the hand or just below the elbow. In some cases the blood is taken from an artery. The skin over the area is wiped with an antiseptic, and an elastic tourniquet is tied around the arm to retain blood within the arm and render the veins more visible. The phlebotomist feels the veins to select an appropriate one, inserts a needle into the vein, and releases the elastic band. The appropriate amount of blood is drawn, and the needle is withdrawn from the vein. While phlebotomy is a relatively simple process, a risk of infection exists. This risk is minimized by using prepackaged sterilized equipment and careful attention to proper technique. Another detrimental effect of phlebotomy is the issue of over sticking the patient. Sometimes, the phlebotomist inserts a needle in multiple areas to locate a cooperative vein. Inserting the needle multiple times is painful for the patient, time consuming for the technician, and uncomfortable for both parties.

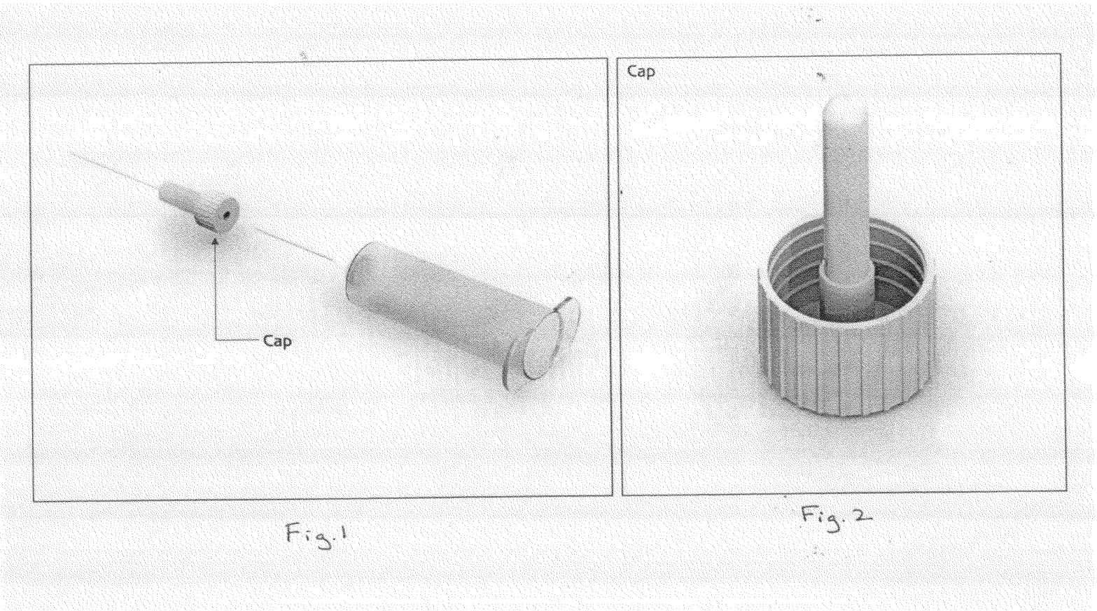

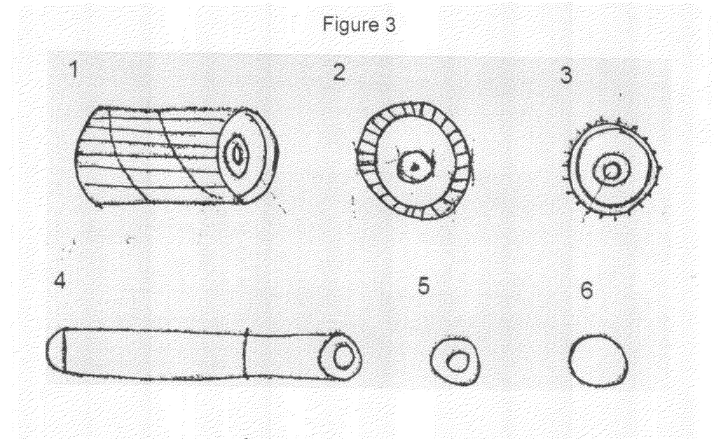

The present invention, hereinafter referred to as the Blood Draw Catheter Top, is a phlebotomy tool specially designed to introduce an intravenous catheter into a blood vessel for blood collection. Technicians use an IV needle to find the vein and extract blood from one central location. The Blood Draw Catheter Top is a sterile clear plastic unit. Although fabricated as one, the Blood Draw Catheter Top is made up of two units or portions (see FIG. 1 and FIG. 2). The first portion resembles a cap, round in shape with a diameter of 10 mm and depth of 8 mm (FIG. 2). This cap portion (FIG. 3, reference 1) is ribbed on the outside for gripping and the interior opening of approximately 8 mm is threaded (FIG. 3, reference 3). In the center of this cap portion is a 1 mm hole for insertion of a needle (FIG. 3, reference 2). The second portion of the Blood Draw Catheter is a tube (FIG. 3, reference 4) approximately 3 cm in length, having a 1 mm opening in one end to house a needle (FIG. 3, reference 5) and closed on the other end (FIG. 3, reference 6). The tube portion extends up from the inside center of the cap portion such that an inserted needle goes through the first cap unit into the tube unit (FIG. 2).

The Blood Draw Catheter Top is placed over the syringe or needle insertion hole in an IV, the latter of which is employed to find a suitable vein. The present invention controls the flow of blood via the interior threads, yet allows it to flow freely enough to collect a sufficient sample.

The Blood Draw Catheter Top facilitates the extraction of blood from one single location as opposed to multiple needle pricks up and down a patient's arms. The Blood Draw Catheter Top improves the working conditions of phlebotomists, as well as lab technicians and nurses. Patients appreciate the present invention's efficient application and can be more comfortable with the necessary blood work.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. The invention claimed is an intravenous, IV catheter or syringe top having a cap and tube portion.

2. The invention claimed is an intravenous, IV catheter or syringe top made of plastic having a cap portion and tube portion, said cap portion being round, being ribbed on the exterior, and having threads on the interior walls with a hole in the center, said tube portion being round with an opening on one end and closed other end, the said tube portion with the open end being congruent with the inside of the said cap portion, such that the hole of the cap portion and the opening of the tube portion match together.

3. The invention claimed is an intravenous, IV catheter or syringe top made of plastic having a cap portion and tube portion, said cap portion being round with a diameter of 1 cm (0.3 in), being ribbed on the exterior, and having threads on the interior walls with a hole of 1 mm in the center, said tube portion being round with a diameter of 3 mm and length of 3 cm with a 1 mm opening on one end and closed other end, the said tube portion with the open end being congruent with the inside of the said cap portion, such that the hole of the cap portion and the opening of the tube portion match together.
